(19) United States
(12) Patent Application Publication
(10) Pub. No.: US 2008/0194840 A1
(43) Pub. Date: Aug. 14, 2008

(54) Process for Preparing Levetiracetam and Racemization of (R)- and (S)-2-Amino Butynamide and the Corresponding Acid Derivatives

(75) Inventors: Arun Kanti Mandal, Vadodara (IN); Satish Wasudeo Mahajan, Vadodara (IN); Pintoo Ganguly, Vadodara (IN); Apurba Chetia, Vadodara (IN); Nitesh Dolatram Chauhan, Vadodara (IN); Nilay Dilipkumar Bhatt, Vadodara (IN); Reenaben Ratansing Baria, Vadodara (IN)

Correspondence Address: MERCHANT & GOULD PC, P.O. BOX 2903, MINNEAPOLIS, MN, 55402-0903, US

(73) Assignee: Rubamin Laboratories Limited, Vadodara (IN)

(21) Appl. No.: 11/910,337

(22) PCT Filed: Jan. 20, 2006
(86) PCT No.: PCT/IN2006/000020

§ 371 Date: Oct. 1, 2007

(30) Foreign Application Priority Data
Apr. 1, 2005 (IN) 412/MUM/2005
May 30, 2005 (IN) 643/MUM/2005

(51) Int. Cl. C07D 207/267

(52) U.S. Cl. 548/550

(57) ABSTRACT

Process for the preparation of (S)-(−)-α-ethyl-2-oxo-1-pyrrolidineacetamide of Formula (I), comprising the steps of (i) resolution of racemic 2-amino butynamide with L-(+)-tartaric acid either in alcoholic solvents like methanol, isopropanol, ethanol or in water or mixture of water-alcohol to provide (S)-(+)-2-amino butynamide tartarate salt; and ii) direct conversion of (S)-(+)-2-amino butynamide tartarate salt and 4-halobutryl chloride in presence of inorganic or organic base in suitable solvent and drying agents yielded the desired (S)-(−)-α-ethyl-2-oxo-1-pyrrolidineacetamide (I). Further (S)-(+)-2-amino butynamide tartarate salt is converted to (S)-(+)-2-amino butynamide hydrochloride salt, by reacting with an inorganic or organic base in a suitable solvent followed by reaction with HCl gas in an appropriate solvent. The preparation of (S)-(+)-2-amino butynamide hydrochloride salt, which is an intermediate for Levetiracetam, is prepared from (S)-(+)-2-amino butynamide tartarate salt in presence of inorganic base selected from potassium carbonate or hydroxide, sodium carbonate or hydroxide, ammonia gas, and organic base selected from triethyl amine, DMAP, and the like and a suitable solvent selected from methanol, isopropanol, ethanol or in water or mixture of water-alcohol.

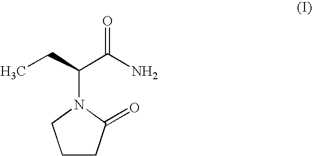

### FIELD OF INVENTION

The present invention relates to a new process for preparing (S)-(−)-α-ethyl-2-oxo-1-pyrrolidine acetamide represented by the following formula (I)

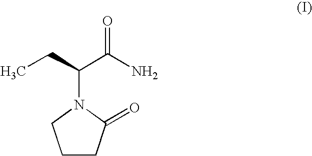

The compound is called Levetiracetam, and is known to be useful as an agent for the treatment or prevention of epilepsy and other neurological disorders.

### BACKGROUND OF INVENTION

British Pat. No. 1,309,692 teaches the synthesis of (S)-(−)-α-ethyl-2-oxo-1-pyrrolidine acetamide of formula (I) The prior art methods for synthesis of Compound (I) could be summarised as follows:

U.S. Pat. No. 4,696,943 (Gobert et al.) describes the method either by reacting (S)-(−)-α-ethyl-2-oxo-1-pyrrolidineacetic acid successively with an alkyl chloro formate and with ammonia or by condensation followed by cyclization of 2-amino butanamide with 4-chlorobutyryl chloride. This process requires starting reactant with correct stereochemical configuration, the yields are often poor in the resolution.

GB 2225322 (Cossement E. et al.) claims (S)-(−)-α-ethyl-2-oxo-1-pyrrolidine acetamide preparation by hydrogenolysis of (S)-α-[2-(methylthio)ethyl]-2-oxo-1-pyrrolidineacetamide in the presence of a desulfurising reagent. In this process the desulfurising agents are not environment friendly.

U.S. Pat. No. 6,107,492 (Futagawa et al.) describes the method by optical resolution of racemic α-ethyl-2-oxo-1-pyrrolidineacetamide by means of preparative high performance liquid chromatography or continuous simulated moving bed chromatographic system using silica gel supported amylose tris (3,5-dimethylphenyl carbamate) as a packing material. U.S. Pat. No. 6,124,473 (Cavoy et al.) claims an industrial scale enantiomeric resolution of racemic mixture of α-ethyl-2-oxo-1-pyrrolidineacetamide by simulated moving bed chromatography, using at least three columns filled with chiral stationary phase.

EP 1477478 (Surtees et al.) describes a process for preparing α-ethyl-2-oxo-1-pyrrolidineacetamide from lactam substituted 2-butanoic acid derivatives based on similar methodologies adopted by Boaz et al in U.S. Pat. No. 6,686,477 which involves preparation of enantiomerically pure lactum substituted propanoic acid derivatives by asymmetric hydrogenation of lactam substituted 2-propenoic acid derivatives. The disadvantage of the process is the reaction time necessary to obtain the conversion is very long and hence not attractive.

WO 03/014080 (Ates et al.) claims an improved process for (S)-(−)-α-ethyl-2-oxo-1-pyrrolidineacetamide from (S)-(+)-2-aminobutyric acid by allylation of its methyl ester with ethyl-4-bromobutyrate, cyclization and amidation. Here again expensive optical active reactant is required.

WO 2004/069796 (Dolityzky) describes a process for preparing (S)-(−)-α-ethyl-2-oxo-1-pyrrolidineacetamide from (S)-(+)-2-amino butynamide hydrochloride with 4-chlorobutyryl chloride in Acetonitrile or methyl tert butyl ether in the presence of a strong base. Here also expensive optical active reactant is required.

WO 2004/076416 (Surroca et al.) describes a method which comprises of preparation of aminomethyl derivatives of racemic α-ethyl-2-oxo-1-pyrrolidineacetamide, resolution followed by deaminomethylation of sufficiently pure enantiomeric intermediate to make (S)-(−)-α-ethyl-2-oxo-1-pyrrolidineacetamide. The loss during resolution males this process unattractive

### OBJECT OF THE INVENTION

It is an object of the invention to provide a new, short, cost effective process for the preparation of (S)-(−)-α-ethyl-2-oxo-1-pyrrolidineacetamide, also known as Levetiracetam, in high yields.

It is another object of the present invention to make the process more economically viable and environment friendly by converting the byproduct in this process namely (R)-2-amino butynamide (1a) to its racemic form, i.e. (RS)-2-amino-butynamide (2a) and then resolving to produce the desired (S)-2-amino butynamide (3a) and recycled on a continuous basis.

In our copending application No. 264/MUM/2005 there is disclosed a process for the preparation of (S)-(−)-α-ethyl-2-oxo-1-pyrrolidineacetamide of Formula (I) in which the step of resolution is omitted. The process of the copending application comprises:

i) condensation of (S)-2-amino butanol of Formula (II) and 4-halobutryl chloride, where halo group can be chloro, bromo or iodo in solvents to form α-ethyl-2-oxo pyrrolidine ethanol of Formula (III)

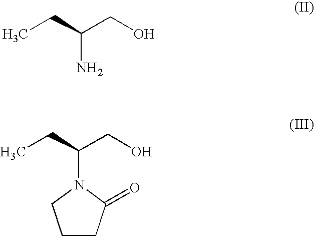

ii) oxidation of (S)-α-ethyl-2-oxo pyrrolidine ethanol to yield (S)-α-ethyl-2-oxo pyrrolidine acetic acid having the formula (IV)

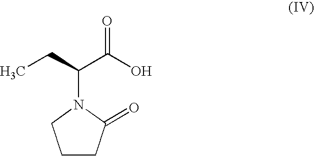

iii) esterification of (S)-α-ethyl-2-oxo pyrrolidine acetic acid (IV) with an alcohol to provide alkyl ester of Formula (V) wherein, R is 1-4 Carbon atom.

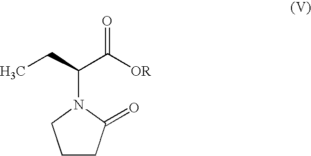

iv) ammonolysis of alkyl esters of formula (V) with ammonia to provide (S)-(−)-α-ethyl-2-oxo-1-pyrrolidine acetamide of formula (I).

### SUMMARY OF INVENTION

The present invention relates to a process for the preparation of (S)-(−)-α-ethyl-2-oxo-1-pyrrolidineacetamide of Formula (I), comprising the steps of:

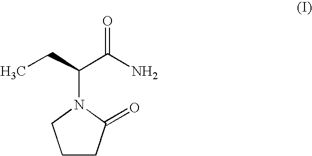

i) resolution of racemic 2-amino butynamide with L-(+)-tartaric acid either in alcoholic solvents like methanol, isopropanol, ethanol or in water or mixture of water-alcohol to provide (S)-(+)-2-amino butynamide tartarate salt (VI).
ii) direct conversion of (S)-(+)-2-amino butynamide tartarate salt (VI) and 4-halobutryl chloride in presence of inorganic or organic base in suitable solvent and drying agents yielded the desired (S)-(−)-α-ethyl-2-oxo-1-pyrrolidineacetamide (I).

The advantage of this route is that it allows synthesis of (S)-(−)-α-ethyl-2-oxo-1-pyrrolidineacetamide (I) in one step from (VI) and does not require prior isolation of the intermediate (S)-(+)-2-amino butynamide. Hydrochloride salt, which is a common practice after resolution before its conversion to (I). This therefore cuts short the synthesis by one step.

If desired, (S)-(+)-2-amino butynamide tartarate salt (VI) can also be converted to (S)-(+)-2-amino butynamide hydrochloride salt, by reacting with an inorganic or organic base in a suitable solvent followed by reaction with HCl gas in an appropriate solvent. (S)-(+)-2-amino butynamide hydrochloride thus formed, can be converted to Levetiracetam of formula (I) using processes described in prior art.

According to another aspect of the invention there is provided a process for the preparation of (RS)-2-amino butyric acid derivatives ((2a-c) comprising racemization of optical active (R)-2-amino butyric acid derivatives (1a-c) or (S)-2-amino butyric acid derivatives (3a-c) to convert to (RS)-2-amino butyric acid derivatives ((2a-c).

If desired the same methodology can be used to convert (S)-2-amino butynamide (3a) or (S)-2-amino butyric acid derivatives (3a-c) to (RS)-amino butynamide (2a), or the corresponding acid derivatives.

The racemisation can be done in the presence of a wide range of bases, alone or in combination, in polar solvents and at a wide range of temperature and pressures.

### DETAILED DESCRIPTION OF THE INVENTION

The (S)-(+)-2-amino butynamide tartarate salt, used in this invention is obtained from the racemic (±)-2-amino butynamide by chemical resolution with (L)-(+)-tartaric acid. The amount of (L)-(+)-tartaric acid used could vary from 0.25 molar amount to 1 molar amount. The isolation of the tartarate salt formed by successive crystallizations either in alcoholic solvents like methanol, isopropanol, ethanol or in water or mixture of water and alcohol. The crystallization can be effected at temperature between 25° C. to 60° C., but preferably between 40° C. to 50° C.

The preparation of (S)-(−)-α-ethyl-2-oxo-1-pyrrolidineacetamide of Formula (I),

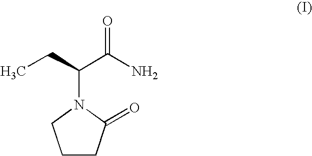

comprises reaction of (S)-(+)-2-amino butynamide tartarate salt and 4-chlorobutyryl chloride in presence of inorganic base selected from potassium carbonate or hydroxide, sodium carbonate or hydroxide, ammonia or organic base selected from triethyl amine, DMAP, DABCO and the like.

The reaction can be carried out in the presence of tetraalkyl ammonium halide (R₄N⁺X), R can be C1 to C4 carbon atom and or Benzyl trialkyl ammonium halides, where alkyl group could be Carbon 1 to 4 atom.

The suitable solvent used is aprotic solvent selected from chlorinated solvents, such as methylene chloride, chloroform, carbon tetrachloride, dichloroethane and others like Acetonitrile, Dimethyl formamide (DMF), Methyl t-butyl ether and Tetrahydrofuran. The solvent can also be chosen from aromatic hydrocarbon solvent, like toluene, xylene, mixed xylenes and like.

The drying agents are selected from Sodium sulphate, Magnesium sulphate and molecular sieve. The temperature of the reaction is between 0 to 40° C., preferably between 0 to 5° C.

(S)-(+)-2-amino butynamide hydrochloride salt, which is an intermediate for Levetiracetam, is prepared from (S)-(+)-2-amino butynamide tartarate salt in presence of inorganic or organic base. The inorganic base is selected from potassium carbonate or hydroxide, sodium carbonate or hydroxide, ammonia gas. The Organic base is selected from triethyl amine, DMAP, and the like. The suitable solvent is selected from methanol, isopropanol, ethanol or in water or mixture of water-alcohol.

The hydrochloride salt is prepared by passing HCl gas directly, or using a preformed solution of HCl gas in alcoholic solvent like Isopropanol, Methanol, Ethanol, propanol etc to the solution of (S)-(+)-2-amino butynamide in the chosen solvent. The temperature during the reaction is maintained between 0-40° C., preferably between 20-30° C.

The process according to the preferred aspect of the invention enables continuous recycle of (S)-2-amino butynamide (3a) from the unwanted (R)-2-amino butynamide (1a), thereby making the process for the manufacture of (S)-2-amino butynamide (3a) more economically viable and environment friendly. The process is shown in scheme I below

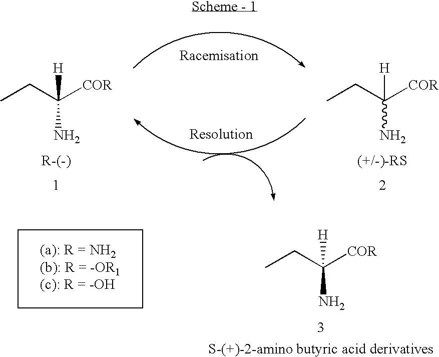

The base can be used in catalytic quantity (5-20 mole percent range) or can be used stoichiometrically to effect the racemisation. The temperature of reaction can be from a range of 30-100° C. The preferred temperatures range being 60 to 110° C. The reaction can be carried out at ambient pressure (1 atm) to high pressure upto 20 atmosphere, the preferable pressure being 5-12 atmosphere.

The time required for the racemisation varies from 1 hour to 72 hours depending upon the choice of the base, solvents, temperature and pressure used in the reaction to effect complete racemization.

The process of the invention is provided hereunder in greater detail in relation to non-limiting examples hereunder:

### EXAMPLE-1

### 1) Preparation of Tartarate Salt of (S)-amino Butynamide

102 gm racemic (±)2-amino butynamide is suspended in 1400 ml of methanol in a 3-liters of round bottom flask. To this suspension is added gradually 150 gm of L(+) tartaric acid under stirring at 25° C. The mixture is then heated to reflux. After 30 minutes refluxing, salt is precipitated. It is then cooled to 40-50° C., filtered to get 83 g of (S)-(+)-amino butynamide tartarate salt.

Yield: 83 g, Appearance: white solid, [α]²⁵_{D}+280 (C=1, water).

### 2) Preparation of (S)-alpha-ethyl-2-oxo-1-pyrrolidineacetamide (I)

33.8 g of anhydrous Na₂SO₄ is added to suspension of 50 g of (S)-amino butynamide tartarate salt in 1 liter of Acetonitrile. The mixture is cooled to 0-5° C., 82 g of potassium carbonate is added to it. A solution of (30.7 g) of 4-chloro butyryl chloride in 90 ml acetonitrile is added dropwise at 0° C. with vigorous stirring. After the addition, the reaction mixture is allowed to return to 25° C. After 5 hours' cool the reaction mixture, 38.8 g powdered potassium hydroxide is added at 0-5° C. The reaction mixture was stirred for further 10 his, filtered and filtrate evaporated under reduce pressure. The residue (28 g, 90% pure) is dissolved in 130 ml of Acetone and heated to reflux temperature and filtered hot. After cooling the filtrate, the desired product crystallizes to give 22.6 g of (S)-alpha-ethyl-2-oxo-1-pyrrolidineacetamide.

Melting point: 115-117° C.; [α]²⁵_{D} −88° (C=1, acetone), Yield: 67%

### 3) Preparation of (S)-(+)-2-amino Butynamide Hydrochloride

150 g (S)-amino butynamide tartarate salt is added in 750 ml of Methanol. The mixture is cooled to 20-30° C. Ammonia gas is passed in to the solution keeping the temperature 20-30° C. till the pH1 of reaction mass becomes 9-10. The reaction mixture was stirred for 30 minutes and filtered. Methanol is removed, 50 ml of isopropanol was added and the reaction mixture was acidified to pH 2 by adding mixture of hydrochloric acid gas in Isopropanol maintaining the temperature between 20-30° C. The solid filtered, and dried to obtain 58 g of (S)-(+)-2-amino butynamide hydrochloride.

[α]²⁵_{D} 26.5° (C=1, methanol), Yield: 70%

### EXAMPLE-2

In to a autoclave with stirrer were charged 75 g of (R)-2-amino butynamide and 300 ml methanol. The autoclave was sealed and 2 kg/cm² ammonia gas was charged. Heated the mixture to 100° C. for 72 hours. The reactor was cooled to 25° C. Methanol distilled off and the product collected.

Yield 23.5 g, [α]_{D}²⁵ −2.172° c=1, methanol

### EXAMPLE-3

50 g (S)-amino butynamide hydrochloride is added in 150 ml of Methanol. 39 g of Sodium methoxide was added to it. Stirred for 4 hours. The reaction mixture was filtered. The filtrate was distilled under reduce pressure. The pH of the reaction mixture was adjusted to 2 by 15% Isopropanol hydrochloric acid mixture (180 ml) maintaining the temperature between 20-30° C. Solid was filtered and dried at 50-60° C.

Yield 36 g, [α]²⁵_{D} 0.293° (C=1, methanol)

1. A process for the preparation of (S)-(−)-α-ethyl-2-oxo-1-pyrrolidineacetamide of Formula (I), comprising the steps of: 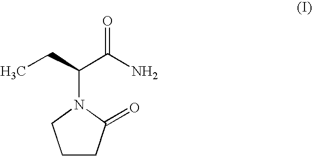
i) resolution of racemic 2-amino butynamide with L-(+)-tartaric acid either in alcoholic solvents like methanol, isopropanol, ethanol or in water or mixture of water-alcohol to provide (S)-(+)-2-amino butynamide tartarate salt; and
ii) direct conversion of (S)-(+)-2-amino butynamide tartarate salt and 4-halobutryl chloride in presence of inorganic or organic base in suitable solvent and drying agents yielded the desired (S)-(−)-α-ethyl-2-oxo-1-pyrrolidineacetamide (I).

2. A process according to claim 1 further comprising converting (S)-(+)-2-amino butynamide tartarate salt to (S)-(+)-2-amino butynamide hydrochloride salt, by reacting with an inorganic or organic base in a suitable solvent followed by reaction with HCl gas in an appropriate solvent.

3. A process according to claim 2 wherein the (S)-(+)-2-amino butynamide hydrochloride is converted to Levetiracetam of formula (I) by known methods.

4. A process according to claim 1 wherein (S)-(+)-2-amino butynamide tartarate salt is obtained from the racemic (±)-2-amino butynamide by chemical resolution with 0.25 molar amount to 1 molar amount (L)-(+)-tartaric acid.

5. A process according to claim 4 wherein the tartarate salt is isolated by successive crystallizations in alcoholic solvents like methanol, isopropanol, ethanol or in water or mixture of water and alcohol.

6. A process according to claim 5 wherein the crystallization is effected at temperature between 25° C. to 60° C., and preferably between 40° C. to 50° C.

7. A process for the preparation of the compound (S)-(−)-α-ethyl-2-oxo-1-pyrrolidineacetamide of Formula (I), 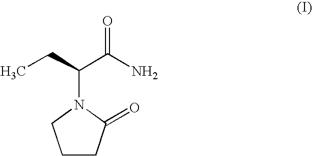
comprising reacting (S)-(+)-2-amino butynamide tartarate salt and 4-chlorobutyryl chloride in presence of inorganic base selected from potassium carbonate or hydroxide, sodium carbonate or hydroxide, ammonia or the organic base selected from triethyl amine, DMAP, DABCO and the like.

8. A process according to claim 7 wherein the reaction can be carried out in the presence of tetraalkyl ammonium halide (R₄N⁺X), R can be C1 to C4 carbon atom and or Benzyl trialkyl ammonium halides, where alkyl group could be Carbon 1 to 4 atom.

9. A process according to claim 7 wherein the solvent is aprotic solvent selected from chlorinated solvents, such as methylene chloride, chloroform, carbon tetrachloride, dichloroethane e; others like Acetonitrile, Dimethyl formamide (DMF), Methyl t-butyl ether and Tetrahydrofuran.

10. A process according to claim 9 wherein the solvent is chosen from aromatic hydrocarbon solvent, like toluene, xylene, mixed xylenes and like.

11. A process according to claim 1 wherein drying agents selected from sodium sulphate, magnesium sulphate and molecular sieve are used.

12. A process according to claim 1 wherein the temperature of the reaction is between 0 to 40° C., preferably between 0 to 5° C.

13. A process for preparation of (S)-(+)-2-amino butynamide hydrochloride salt, which is an intermediate for Levetiracetam, is prepared from (S)-(+)-2-amino butynamide tartarate salt in presence of inorganic base selected from potassium carbonate or hydroxide, sodium carbonate or hydroxide, ammonia gas, and organic base selected from triethyl amine, DMAP, and the like and a suitable solvent selected from methanol, isopropanol, ethanol or in water or mixture of water-alcohol.

14. A process for the preparation of (RS)-2-amino butyric acid derivatives ((2a-c) comprising racemization of optical active (R)-2-amino butyric acid derivatives (1a-c) or (S)-2-amino butyric acid derivatives (3a-c) to convert to (RS)-2-amino butyric acid derivatives ((2a-c).

15. A process for the preparation of (RS)-amino butynamide (2a), or the corresponding acid derivatives comprising racemization of (S)-2-amino butynamide (3a) or (S)-2-amino butyric acid derivatives (3a-c) to convert to (RS)-amino butynamide (2a), or the corresponding acid derivatives.

16. A process of claim 14 or 15 wherein the racemisation is carried out in the presence of a base.

17. A process of claim 16 wherein the racemisation is carried out also in the presence of polar solvents.
